(19) United States
(12) Patent Application Publication
(10) Pub. No.: US 2025/0035616 A1
(43) Pub. Date: Jan. 30, 2025

(54) Organoid Compositions

(71) Applicants: SHIMADZU CORPORATION, Kyoto (JP); OSAKA UNIVERSITY, Osaka (JP)

(72) Inventors: Hiroyuki MIZUGUCHI, Osaka (JP); Yukiko UEYAMA-TOBA, Osaka (JP); Jumpei YOKOTA, Osaka (JP); Yanran TONG, Osaka (JP); Hayato MATSUI, Kyoto-shi (JP); Sachiko KOBAYASHI, Kyoto-shi (JP); Junko ENOMOTO, Kyoto-shi (JP)

(73) Assignees: SHIMADZU CORPORATION, Kyoto (JP); OSAKA UNIVERSITY, Osaka (JP)

(21) Appl. No.: 18/784,235

(22) Filed: Jul. 25, 2024

(30) Foreign Application Priority Data
Jul. 28, 2023 (JP) 2023-123189

(51) Int. Cl. G01N 33/50; C12N 5/071

(52) U.S. Cl. CPC G01N 33/5088; C12N 5/0671; C12N 5/068; G01N 33/5014; C12N 2533/40; G01N 2500/10

(57) ABSTRACT

Provided is a high-function organoid composition. Also provided is a method of producing the organoid composition. The organoid composition is characterized by including: an organoid including a cell cluster of endodermal cells; and an amphiphilic block polymer having a hydrophilic block chain having a sarcosine unit and a hydrophobic block chain having a lactic acid unit. The organoid composition may be produced by culturing the endodermal cells in a culture system containing the amphiphilic block polymer. The organoid composition thus obtained can evaluate pharmacokinetics and drug toxicity, and is hence suitable for drug screening.

### BACKGROUND OF THE INVENTION

### 1. Field of the Invention

The present invention relates to an organoid composition. The present invention also relates to a method of producing the organoid composition.

### 2. Description of the Related Art

In regenerative medicine or drug screening, the function of a cell to be used needs to be improved to a state close to that of a living body, and an organoid culture technology has been attracting attention in recent years. An organoid is a three-dimensionally cultured (3D cultured) cell having a feature and proliferation potency similar to those of a three-dimensionally formed organ, and has been expected to, for example, find application as an in vitro organ model in a field, such as cell research, drug discovery screening, or regenerative medicine, for creating each tissue or organ in a living body from a cell. It has been said that the three-dimensionally formed organoid has an excellent function close to that of a living body as compared to a two-dimensionally cultured (2D cultured) cell. For example, a human liver organoid is a three-dimensional histological body, which is created by three-dimensional culture based on the development and regeneration mechanism of an organ, and shows the anatomical and function scientific features of an in vivo organ, and hence the organoid has been expected as a novel cell source conducive to drug discovery research and regenerative medicine (Huch et al., Nature, 2013 Feb. 14; 494(7436): 247-50).

For example, Matrigel (trademark) (Corning) that is a culture substrate optimum for organoid culture contains an animal-derived component, and hence a variation between batches is large. Accordingly, concern is raised about the reproducibility of an experiment at the time of the application of an organoid technology to drug discovery. In addition, at the time of the application thereof to regenerative medicine, there is a problem in terms of safety because concern is raised about the risk of an infection or an immune reaction. Meanwhile, a synthetic polymer is expected to serve as a material overcoming the problems of the Matrigel to contribute to drug discovery research and regenerative medicine because of, for example, its high purity, high reproducibility, controlled chemical composition, and mechanical characteristics.

The inventors of the present invention have developed an amphiphilic block polymer having polysarcosine in its hydrophilic portion and polylactic acid in its hydrophobic portion (Matsui et al., Mater. Today Commun., 2017, 156-162 and WO 2017/017969 A1 (Japanese Patent No. 6354905)). In WO 2018/142633 A1 (Japanese Patent No. 6711424), there is a disclosure that a cell was cultured with the amphiphilic block polymer. Further, it has been reported that the application of the amphiphilic block polymer to a human induced pluripotent stem (iPS) cell-derived liver cell enables the production of a liver spheroid (J. Enomoto et al., ACS Applied Bio Materials, 2021, Sep. 20; 4(9): 7290-7299).

In Japanese Patent Application Laid-open No. 2018-113945, as a method of performing three-dimensional culture through utilization of a scaffold, there is a disclosure of a method including: bringing a gelatin solution and a cell into contact with each other; and cooling the resultant to cause the gelatin to gel, to thereby form a spheroid in a short time period. In Japanese Patent Translation Publication No. 2022-546845, there is a disclosure of a method of producing a human enterocyte-derived organoid through use of a multi-arm PEG having an ethylenically unsaturated group.

### SUMMARY OF THE INVENTION

An object of the present invention is to provide a high-function organoid composition. Another object thereof is to provide a method of producing the organoid composition.

To achieve the above-mentioned objects, the inventors of the present invention have attempted the establishment of a technology for the culture of a high-function organoid composition, and have made extensive investigations. As a result, the inventors have succeeded in producing a high-function organoid composition through the culture of an endodermal cell in a culture system containing an amphiphilic block polymer having a hydrophilic block chain and a hydrophobic block chain. The organoid composition provides an organoid composition that can evaluate pharmacokinetics and drug toxicity, and is hence suitable for drug screening. Thus, the inventors have completed the present invention.

That is, the present invention includes the following.

1. An organoid composition, including: an organoid including a cell cluster of endodermal cells; and an amphiphilic block polymer present in the cell cluster for forming the organoid, the polymer having a hydrophilic block chain having a sarcosine unit and a hydrophobic block chain having a lactic acid unit.

2. The organoid composition according to the above-mentioned item 1, wherein the amphiphilic block polymer has the hydrophilic block chain having the 20 or more sarcosine units and the hydrophobic block chain having the 10 or more lactic acid units.

3. The organoid composition according to the above-mentioned item 1, wherein the endodermal cells are one of liver cells or intestinal cells.

4. The organoid composition according to the above-mentioned item 3, wherein the liver cells are one of liver cells collected from a living body or pluripotent stem cell-derived liver cells.

5. The organoid composition according to the above-mentioned item 3, wherein the intestinal cells are one of intestinal epithelial cells collected from a living body or pluripotent stem cell-derived intestinal epithelial cells.

6. An organoid composition for evaluating pharmacokinetics and/or evaluating drug toxicity, including the organoid composition of the above-mentioned item 1.

7. A method of producing the organoid composition of the above-mentioned item 1, including the following steps:
(1) a step of dispersing endodermal cells; and
(2) a step of culturing the dispersed cells in a culture system containing an amphiphilic block polymer having a hydrophilic block chain having a sarcosine unit and a hydrophobic block chain having a lactic acid unit.

8. The production method according to the above-mentioned item 7, wherein the step of dispersing the endodermal cells serving as the step (1) is a step of dispersing the endodermal cells that have three-dimensionally aggregated.

9. The production method according to the above-mentioned item 8, wherein the endodermal cells are one of liver cells or intestinal cells.

10. A kit for evaluating pharmacokinetics and/or evaluating drug toxicity, including: the organoid composition of any one of the above-mentioned items 1 to 6; and a device and/or a reagent required for a pharmacokinetic evaluation and/or a drug toxicity evaluation or inspection.

11. A method of evaluating pharmacokinetics and/or a method of evaluating drug toxicity, including using the organoid composition of any one of the above-mentioned items 1 to 6.

The organoid composition according to at least one embodiment of the present invention can be effectively utilized in the evaluation of pharmacokinetics because the composition shows high drug-metabolizing enzyme activity. Further, the organoid composition according to at least one embodiment of the present invention shows excellent sensitivity to a drug causing hepatotoxicity. In particular, the organoid composition according to at least one embodiment of the present invention is excellent in safety because the composition is produced through the culture in the culture system containing the amphiphilic block polymer having the hydrophilic block chain and the hydrophobic block chain.

### BRIEF DESCRIPTION OF THE DRAWINGS

FIG. 1A is an illustration of a protocol for the production of a liver organoid composition including HYDROX. FIG. 1B shows the results of the observation of the liver organoid composition including the HYDROX with a phase-contrast microscope (Example 1).

FIG. 2 shows the results of the measurement of the expression levels of liver cell marker genes in the liver organoid composition including the HYDROX (Example 1).

FIG. 3 shows the results of the measurement of the enzyme activities of drug-metabolizing enzymes in the liver organoid composition including the HYDROX (Example 1).

FIG. 4 shows the results of the hepatotoxicity evaluation of the liver organoid composition including the HYDROX (Example 1).

FIG. 5A shows the results of the observation of an intestinal organoid composition including the HYDROX with a phase-contrast microscope. FIG. 5B shows the results of the observation of a Matrigel-cultured intestinal organoid and the intestinal organoid composition including the HYDROX with a transmission electron microscope (Example 2).

FIG. 6A shows the results of the measurement of the expression levels of intestinal differentiated cell marker genes in the intestinal organoid composition including the HYDROX.

FIG. 6B shows the results of the measurement of the enteroendocrine cell marker of the intestinal organoid composition including the HYDROX (Example 2).

FIG. 7A shows the results of the measurement of the expression levels of pharmacokinetics-related genes in the intestinal organoid composition including the HYDROX. FIG. 7B shows the results of the measurement of the enzyme activity of CYP3A4 in the intestinal organoid composition including the HYDROX (Example 2).

FIG. 8 shows the results of the measurement of the expression levels of the pharmacokinetics-related genes in the liver organoid composition including the HYDROX and a two-dimensionally cultured endodermal cell-derived amphiphilic block polymer-cultured liver organoid (Example 3).

FIG. 9 shows the recognition of the coexistence of the HYDROX and the respective organoid compositions by NMR (Example 4).

### DESCRIPTION OF THE EMBODIMENTS

The present invention relates to an organoid composition characterized by including: an organoid including a cell cluster of endodermal cells; and an amphiphilic block polymer present in the cell cluster for forming the organoid, the polymer having a hydrophilic block chain having a sarcosine unit and a hydrophobic block chain having a lactic acid unit. The present invention also relates to a method of producing the organoid composition.

The organoid composition according to at least one embodiment of the present invention includes the organoid including the cell cluster of the endodermal cells. The term “organoid” as used herein refers to such a cell cluster that the cells form a cluster in a culture container. The organoid may or may not hold a culture carrier for holding a three-dimensional structure, and may be such a cell cluster that the cells are bonded to each other under a floating state in the culture container. The term “endodermal cell” refers to a cell for forming, for example, a liver, intestines (e.g., a small intestine and a large intestine), a stomach, a pancreas, a pharynx, a trachea, a bronchus, a lung, a bladder, or a urethra. In particular, a cell for forming a liver (hereinafter referred to as “liver cell” or “hepatocyte”) or a cell for forming intestines (hereinafter referred to as “intestinal cell”) is applied as the “endodermal cell.”

The endodermal cells in at least one embodiment of the present invention are endodermal cells collected from a living body or pluripotent stem cell-derived endodermal cells. The endodermal cells collected from the living body are endodermal cells collected by an operation or the like, or a commercial endodermal cell strain (e.g., an endodermal cell strain established from endodermal cells collected in the past by an operation or the like). Specifically, for example, a liver cell is a liver cell collected from the living body or a pluripotent stem cell-derived liver cell. For example, an intestinal cell is an intestinal epithelial cell collected from the living body or a pluripotent stem cell-derived intestinal epithelial cell. The organoid in at least one embodiment of the present invention is an organoid derived from an endodermal cell collected from the living body or a pluripotent stem cell-derived organoid.

### (Method of Producing Organoid)

An organoid produced from a liver cell collected from a living body or a pluripotent stem cell-derived liver cell is referred to as “liver organoid.” The liver organoid produced from the liver cell collected from the living body may be produced from, for example, a fresh or cryopreserved liver tissue, and may be produced by any one of various methods that are known per se or to be developed in the future. The organoid may be produced, for example, by a method described in Examples or through use of HepatiCult™ Organoid Growth Medium (Human) (STEMCELL Technologies). The organoid may be produced as follows: the fresh or cryopreserved liver tissue is turned into a single cell with, for example, a protease, such as trypsin, a collagenase, or Dispase I, EDTA, or EGTA; the cell is recovered and subjected to washing and centrifugation treatment; and then the organoid is produced from the cell by utilizing a substrate for an organoid. The term “substrate for an organoid” as used herein may refer to substrates made of various materials and having various structures, the materials and structures being known per se or to be developed in the future. The substrate is specifically, for example, a solubilized basement membrane extracted from EHS mouse sarcoma rich in an ECM protein containing a hydrogel, laminin (main component), type IV collagen, a heparin sulfate proteoglycan, entactin/nidogen, and various growth factors, and for example, a Matrigel (trademark) (Corning) basement membrane is used. The density of seeded cells only needs to enable organoid formation, and is hence not particularly limited. However, the density is, for example, 4×10⁴ cells/well (24-well plate), preferably 3×10⁴ cells/well (24-well plate), most preferably 2×10⁴ cells/well (24-well plate) The cells may be cultured with a medium for a liver organoid after, for example, their incubation at 37° C. for from 1 minute to 60 minutes, preferably from 1 minute to 30 minutes, more preferably from 10 minutes to 15 minutes.

The medium for a liver organoid only needs to enable the culture of a liver cell, and is hence not particularly limited. However, a medium containing, for example, a minimum essential medium eagle (MEM) or Roswell Park Memorial Institute (RPMI) medium as a main component, and containing, for example, Advanced™ DMEM/F12 (GIBCO) or a medium component described in Meritxell Huch et al., Nature vol. 494, p. 247-250 (2013) may be used as a main medium. Specifically, HepatiCult™ Organoid Growth Medium (Human) (STEMCELL Technologies) may be used. In initial culture, the above-mentioned medium component may be used while antibiotics, such as penicillin G sodium salt, streptomycin sulfate, and amphotericin B, for example, 1×Antibiotic-Antimycotic (Sigma-Aldrich), and a Rho-binding kinase inhibitor such as Y-27632 are each appropriately incorporated thereinto. For example, a medium obtained by incorporating, into Advanced DMEM/F12 (product name), R-spondinl conditional medium (product name), 1×B27 supplement Minus Vitamin A (product name), penicillin/streptomycin, nicotinamide, N-acetyl-L-cysteine, GlutaMAX (product name), 4-(2-hydroxyethyl)-1-piperazineethanesulfonic acid (HEPES), Epidermal Growth Factor (EGF), gastrin, Hepatocyte growth factor (HGF), fibroblast growth factor 10 (FGF10), A83-01, and forskolin may be used. The medium may be appropriately replaced with a new one, and the replacement may be performed, for example, once per 2 to 3 days.

An organoid produced from an intestinal epithelial cell collected from a living body or a pluripotent stem cell-derived intestinal epithelial cell is referred to as “intestinal organoid.” The intestinal organoid produced from the intestinal epithelial cell collected from the living body may be produced from, for example, a fresh or cryopreserved intestinal tissue as in the liver organoid, and may be produced by any one of various methods that are known per se or to be developed in the future. The organoid may be produced, for example, by a method described in Examples or a method described in Japanese Patent Application Laid-open No. 2021-122208 through use of a substrate for an organoid. In this specification, the “intestinal epithelial cell collected from the living body” only needs to be a cell derived from a small intestine (e.g., a duodenum, a jejunum, or an ileum) or a large intestine (e.g., a cecum, a colon, or a rectum), and is hence not particularly limited. However, a cell derived from the small intestine, such as the duodenum, the jejunum, or the ileum, is suitable, and in particular, a cell derived from the duodenum is more suitable.

A medium that may be used in the maintenance and culture of the intestinal organoid only needs to enable the culture of a small intestine tissue-derived cell, and is hence not particularly limited. However, a medium containing Advanced DMEM/F12 as a main component, and containing, for example, a medium component described in Jung et al., Nat. Med. 17, 1225-1227, 2011 or Sato et al., Gastroenterology 141, 1762-1772, 2011 may be used as a main medium. Specifically, IntestiCult™ Organoid Growth Medium (Human) (STEMCELL Technologies) may be used. In initial culture, the above-mentioned medium component may be used while antibiotics, such as penicillin G sodium salt, streptomycin sulfate, and amphotericin B, for example, 1×Antibiotic-Antimycotic (Thermo Fisher Scientific), and a Rho-binding kinase inhibitor such as Y-27632 are each appropriately incorporated thereinto. For example, a medium obtained by incorporating, into Advanced DMEM/F12 (product name), penicillin/streptomycin, HEPES, GlutaMAX (product name), 1×N2 Supplement, 1×B27 Supplement (product name), cysteine, nicotinamide, Afamin-Wnt3A CM, R-Spondinl-CM, A83-01, SB202190, human EGF, human Noggin, or [Leu15]-Gastrin1, or a medium containing Advanced DMEM/F12 as a main component, and containing, for example, a medium component described in Jung et al., Nat. Med. 17, 1225-1227, 2011 or Sato et al., Gastroenterology 141, 1762-1772, 2011 may be used. The medium may be appropriately replaced with a new one, and the replacement may be performed, for example, once per 2 to 3 days.

A pluripotent stem cell-derived organoid may be produced from, for example, induced pluripotent stem cells (iPS cells), and may be produced by any one of various methods that are known per se or to be developed in the future. The organoid may be produced, for example, as follows: an iPS-derived cell (e.g., a pluripotent stem cell-derived liver cell or a pluripotent stem cell-derived intestinal epithelial cell) at each differentiation induction stage is detached from a substrate for iPS cell culture with, for example, a protease, such as trypsin, a collagenase, or Dispase I, EDTA, or EGTA to be turned into a single cell; the cell is recovered and subjected to washing and centrifugation treatment; and then the organoid is produced from the cell by utilizing a substrate for an organoid. A seeding density and the like may be set in the same manner as in the production of an organoid from a liver cell or an intestinal epithelial cell collected from a living body described above.

An organoid can be passaged irrespective of whether the organoid is an organoid derived from an endodermal cell collected from a living body or a pluripotent stem cell-derived organoid. Although a split ratio is not particularly limited, the ratio may be set to, for example, from 1:1 to 1:10. A method obtained by improving an existing method, such as a report by Miyoshi et al. (Miyoshi and Stappenbeck, Nat. Protoc. 8, 2471-2482, 2013), a method described in Sato, T. et al., Gastroenterology. 2011 November; 141(5): 1762-72, or a method described in Sugimoto, S., and Sato, T., Methods Mol Biol. 2017; 1612: 97-105, may be applied as a passage protocol. To passage the organoid, the following is performed: the organoid is suspended in a liquid containing at least any one of, for example, a protease, such as trypsin, a collagenase, or Dispase I, EDTA, and EGTA, for example, TrypLE Select™ (Thermo Fisher Scientific); the suspension is incubated at 37° C. so that its matrix may be degraded; pipetting, centrifugation, and the like are each performed a plurality of times, followed by the removal of a supernatant; and a pellet is resuspended (embedded) in the matrix to a concentration corresponding to the split ratio. The suspension is dropped into a culture carrier and solidified at 37° C., and then the medium is added to the culture carrier. Thus, the passage can be performed. After the passage, until, for example, the second day of culture, the above-mentioned medium component may be used while a Rho-binding kinase inhibitor such as Y-27632 is appropriately incorporated thereinto. In addition, throughout the entire period of the culture, the above-mentioned medium component may be used while antibiotics, such as penicillin G sodium salt, streptomycin sulfate, and amphotericin B, for example, 1×Antibiotic-Antimycotic (Sigma-Aldrich) are each appropriately incorporated thereinto.

Although the kind of the organoid in at least one embodiment of the present invention is not particularly limited, a human organoid is suitable. The organoid in at least one embodiment of the present invention may be cryopreserved with a cryopreservation medium. The cryopreservation of the organoid may be performed by a method known per se. Methods known per se or various methods that are to be developed in the future may each be applied as a method of thawing the cryopreserved organoid.

### (Organoid Composition)

In at least one embodiment of the present invention, the term “organoid composition” refers to a composition further including a polymer component or the like in a cell cluster for forming an organoid. The organoid composition according to at least one embodiment of the present invention is characterized by including, in the cell cluster for forming the organoid, the amphiphilic block polymer having the hydrophilic block chain having the sarcosine unit and the hydrophobic block chain having the lactic acid unit (hereinafter sometimes referred to as “amphiphilic block polymer”). The amphiphilic block polymer having the hydrophilic block chain having the sarcosine unit and the hydrophobic block chain having the lactic acid unit is, for example, HYDROX (trademark) (Shimadzu Corporation). The amphiphilic block polymer is described later. The organoid composition according to at least one embodiment of the present invention includes the amphiphilic block polymer, and hence an unknown component of an animal-derived component is hardly included, and a difference between batches hardly occurs as compared to an organoid utilizing a substrate for an organoid containing the animal-derived component (e.g., an organoid produced by using Matrigel or the like). Further, the gel itself of the substrate for an organoid containing the animal-derived component is difficult to handle and stably produce. Meanwhile, the amphiphilic block polymer in the organoid composition according to at least one embodiment of the present invention is a biodegradable synthetic polymer. Accordingly, when the polymer is utilized as a scaffold substance for an organoid, the composition can be applied to a living body such as a human, and can be applied to a tissue culture field for regenerative medicine. The organoid composition according to at least one embodiment of the present invention is characterized in that the composition shows properties close to those of the living body as compared to an organoid including the animal-derived component. Accordingly, the organoid composition according to at least one embodiment of the present invention is excellent in product quality stability and safety.

The organoid composition according to at least one embodiment of the present invention may be produced by a method including the following steps:
(1) a step of dispersing endodermal cells; and
(2) a step of culturing the dispersed cells in a culture system containing an amphiphilic block polymer having a hydrophilic block chain having a sarcosine unit and a hydrophobic block chain having a lactic acid unit.

The step of dispersing the endodermal cells serving as the step (1) may be any one of various steps that are known per se or to be developed in the future, and is hence not particularly limited. However, the cells may be specifically dispersed, for example, as follows: the cells are suspended in a liquid containing at least any one of, for example, a protease, such as trypsin, a collagenase, or Dispase I, EDTA, and EGTA, for example, TrypLE Select™ (Thermo Fisher Scientific); the suspension is incubated at 37° C.; pipetting, centrifugation, and the like are each performed a plurality of times, followed by the removal of a supernatant; and a cell cluster is suspended in a medium. In the step of dispersing the endodermal cells, for example, a cell strainer may be used. The dispersed cells may be single cells, or may be a small cell cluster.

The step of dispersing the endodermal cells serving as the step (1) is preferably a step of dispersing the endodermal cells that have three-dimensionally aggregated. The organoid composition produced by the method of producing an organoid composition according to at least one embodiment of the present invention is superior to an organoid obtained by a method including a step of dispersing the endodermal cells that have two-dimensionally aggregated, followed by the culture of the cells in the culture system containing the amphiphilic block polymer having the hydrophilic block chain having the sarcosine unit and the hydrophobic block chain having the lactic acid unit (hereinafter referred to as “two-dimensionally cultured endodermal cell-derived amphiphilic block polymer-cultured organoid”) in that an organoid composition having the expression level of a pharmacokinetics-related gene closer to that of a living body is obtained.

Specifically, the organoid composition according to at least one embodiment of the present invention is characterized in that the expression level of a pharmacokinetics-related gene increases as compared to the expression level of the pharmacokinetics-related gene in the two-dimensionally cultured endodermal cell-derived amphiphilic block polymer-cultured organoid. The fact that the expression level of the pharmacokinetics-related gene increases as compared to the expression level of the pharmacokinetics-related gene in the two-dimensionally cultured endodermal cell-derived amphiphilic block polymer-cultured organoid means that for example, when the expression level of the pharmacokinetics-related gene in the two-dimensionally cultured endodermal cell-derived amphiphilic block polymer-cultured organoid is defined as 1, the gene is expressed at a level twice or more, preferably 5 or more times, more preferably 10 or more times, most preferably 50 or more times as high as the foregoing.

The pharmacokinetics-related gene includes a gene encoding a drug-metabolizing enzyme or a transporter. Specific examples thereof include one or a plurality of enzymes selected from a cytochrome P450 (CYP), a UDP-glucuronosyltransferase (UGT), a carboxylesterase (CES), an alcohol dehydrogenase, an aldehyde dehydrogenase, a glutathione peroxidase, a superoxide dismutase, a monoamine oxidase, a diamine oxidase, an epoxide hydrase, an esterase, an amidase, a glutathione S-transferase, a γ-glutamyl transpeptidase, an acetyltransferase, a sulfotransferase, an enzyme involved in a drug transporter, a drug transporter, a peptide transporter, a glucose transporter, a nucleic acid transporter, a cholesterol transporter, and a calcium transporter.

Examples of the CYP include CYP3A4, CYP1A2, CYP2B6, CYP2C8, CYP2C9, CYP2C19, CYP2D6, and CYP2E1. Of those, CYP3A4, CYP1A2, CYP2C8, CYP2C9, and CYP2C19 are preferred, and CYP3A4 is most preferred. Examples of the UGT include UGT1A1, UGT1A3, UGT1A4, UGT1A6, UGT1A6, UGT1A9, UGT2B4, UGT2B7, UGT2B10, UGT2B11, and UGT2B15. Of those, UGT1A1, UGT1A6, UGT2B4, UGT2B7, and UGT2B15 are preferred, and UGT1A1 is most preferred. Examples of the CES include CES2, CES1, CES3, CES4, CES7, and CES8. Of those, CES1 and CES2 are preferred, and CES2 is most preferred. Examples of the drug transporter include Multiple drug resistance 1 (MDR1), breast cancer resistance protein (BCRP), Multidrug resistance-associated Protein 1 (MRP1), Multidrug resistance-associated Protein 2 (MRP2), Multidrug resistance-associated Protein 3 (MRP3), organic cation transporter 1 (OCT1) and organic anion transporting polypeptide 2B1 (OATP2B1). Of those, MDR1 and BCRP are preferred. The peptide transporter is, for example, peptide transporter 1 (PEPT1).

When the endodermal cells in the organoid composition according to at least one embodiment of the present invention are liver cells, the composition is characterized in that the expression level of a liver cell marker gene increases as compared to the expression level of the liver cell marker gene in the two-dimensionally cultured endodermal cell-derived amphiphilic block polymer-cultured organoid. The fact that the expression level of the liver cell marker gene increases as compared to the expression level of the liver cell marker gene in the two-dimensionally cultured endodermal cell-derived amphiphilic block polymer-cultured organoid means that, for example, when the expression level of the liver cell marker gene in the two-dimensionally cultured endodermal cell-derived amphiphilic block polymer-cultured organoid is defined as 1, the gene is expressed at a level 1.5 or more times, preferably twice or more, more preferably 3 or more times, most preferably 3.5 or more times as high as the foregoing. Examples of the liver cell marker include Albumin (ALB), Hepatocyte nuclear factor 1-alpha (HNF1a), Hepatocyte nuclear factor 4-alpha (HFN4a), and Na⁺-taurocholate co-transporting polypeptide (NTCP). The liver cell marker is one or a plurality of markers selected from those markers.

The organoid composition according to at least one embodiment of the present invention may be produced by the step of culturing the dispersed cells in the culture system containing the amphiphilic block polymer having the hydrophilic block chain having the sarcosine unit and the hydrophobic block chain having the lactic acid unit. The phrase “culturing the dispersed cells in the culture system containing the amphiphilic block polymer having the hydrophilic block chain having the sarcosine unit and the hydrophobic block chain having the lactic acid unit” means, for example, the following: the amphiphilic block polymer having the hydrophilic block chain having the sarcosine unit and the hydrophobic block chain having the lactic acid unit is added to a culture carrier, and a suspension containing the dispersed cells is further added thereto, followed by the culture of the cells; or after a solvent has been added to the amphiphilic block polymer, the suspension containing the dispersed cells is added thereto, and the cells are cultured. The dispersed cells may be cultured with a culture carrier to which the amphiphilic block polymer has been applied in advance. In this specification, the solvent is preferably a liquid that does not do damage to the cells, and examples thereof include water, distilled water, physiological saline, a buffer, and a medium.

### (Amphiphilic Block Polymer)

The hydrophilic block chain preferably includes 20 or more sarcosine units (N-methylglycine units). Sarcosine has high water solubility. In addition, polysarcosine has an N-substituted amide, and hence can be subjected to cis-trans isomerization. In addition, the polysarcosine has high flexibility because steric hindrance around α-carbon is small. Accordingly, the use of a polysarcosine chain as a constituent unit results in the formation of a hydrophilic block chain having both of high hydrophilicity and flexibility.

When the number of the sarcosine units of the hydrophilic block chain is 20 or more, the hydrophilic blocks of the molecules of the block polymer present adjacent to each other easily aggregate, and hence gel having incorporated thereinto a hydrophilic dispersion medium, such as water or an alcohol, is easily formed. The upper limit of the number of the sarcosine units in the hydrophilic block chain is not particularly limited. From the viewpoint of causing the hydrophilic blocks of the molecules of the amphiphilic block polymer present adjacent to each other to aggregate to stabilize the structure of the gel, the number of the sarcosine units in the hydrophilic block chain is preferably 300 or less. The number of the sarcosine units is more preferably 25 or more and 200 or less, still more preferably 30 or more and 150 or less.

All the sarcosine units of the hydrophilic block chain may be continuous, or the sarcosine units may be discontinuous as long as the characteristics of the polysarcosine described above are not impaired. When the hydrophilic block chain has a monomer unit except sarcosine, the monomer unit except sarcosine, which is not particularly limited, is, for example, a hydrophilic amino acid or an amino acid derivative. The amino acid includes an α-amino acid, a β-amino acid, and a γ-amino acid. Of those, an α-amino acid is preferred. Examples of the α-amino acid that is hydrophilic include serine, threonine, lysine, aspartic acid, and glutamic acid. In addition, the hydrophilic block may have a sugar chain, polyether, or the like. The hydrophilic block preferably has a hydrophilic group such as a hydroxy group at a terminal thereof (terminal on a side opposite to a linker portion with the hydrophobic block).

The hydrophobic block chain preferably includes 10 or more lactic acid units. Polylactic acid has excellent biocompatibility and excellent stability. In addition, the polylactic acid has excellent biodegradability, and hence the acid is quickly metabolized and has a low accumulation property in a living body. Accordingly, the amphiphilic block polymer using the polylactic acid as a constituent block is useful in its application to the living body, in particular, a human body. In addition, the polylactic acid is crystalline, and hence even when the hydrophobic block chains of block polymer molecules are short, the hydrophobic block chains aggregate in a solvent such as an alcohol. Thus, gel is easily formed.

Although the upper limit of the number of the lactic acid units in the hydrophobic block chain is not particularly limited, the number is preferably 1,000 or less from the viewpoint of stabilizing the structure of the chain. The number of the lactic acid units in the hydrophobic block chain is preferably 10 or more and 1,000 or less, more preferably 15 or more and 500 or less, still more preferably 20 or more and 100 or less.

The lactic acid unit for forming the hydrophobic block chain may be L-lactic acid or D-lactic acid. In addition, L-lactic acid and D-lactic acid may be mixed. All the lactic acid units of the hydrophobic block chain may be continuous, or the lactic acid units may be discontinuous. A monomer unit except lactic acid in the hydrophobic block chain is not particularly limited, and examples thereof include hydroxy acids, such as glycolic acid and hydroxyisolactic acid, and hydrophobic amino acids, such as glycine, alanine, valine, leucine, isoleucine, proline, methionine, tyrosine, tryptophan, glutamic acid methyl ester, glutamic acid benzyl ester, aspartic acid methyl ester, aspartic acid ethyl ester, and aspartic acid benzyl ester, or amino acid derivatives thereof.

### (Structure of Amphiphilic Block Polymer and Synthesis Method Therefor)

The amphiphilic block polymer is obtained by bonding the hydrophilic block chain having the sarcosine unit and the hydrophobic block chain having the lactic acid unit to each other. The hydrophilic block chain and the hydrophobic block chain may be bonded to each other through a linker. A linker having a functional group (e.g., a hydroxy group or an amino group) that can be bonded to a lactic acid monomer (lactic acid or lactide) or a polylactic acid chain, which is a constituent unit for the hydrophobic block chain, and a functional group (e.g., an amino group) that can be bonded to a sarcosine monomer (e.g., sarcosine or N-carboxysarcosine anhydride) or polysarcosine, which is a constituent unit for the hydrophobic block chain, is preferably used as the linker. Appropriate selection of the linker enables the control of the branched structure of the hydrophilic block chain and the hydrophobic block chain.

A method of synthesizing the amphiphilic block polymer is not particularly limited, and for example, a known peptide synthesis method, polyester synthesis method, or depsipeptide synthesis method may be used. More specifically, the amphiphilic block polymer may be synthesized with reference to, for example, WO 2009/148121 A1.

To adjust, for example, the hardness, stability, and degradability (solubility) of gel, the chain length (number of the lactic acid units) of the hydrophobic block chain, and a ratio between the chain lengths (ratio between the number of the lactic acid units and the number of the sarcosine units) of the hydrophobic block chain and the hydrophilic block chain are preferably adjusted. To facilitate the control of the chain length of the hydrophobic block chain, at the time of the synthesis of the amphiphilic block polymer, the following is preferably performed: the hydrophobic block chain (e.g., polylactic acid) having the linker introduced into one terminal thereof is synthesized in advance, and then the hydrophilic block chain (e.g., polysarcosine) is introduced. The chain lengths of the hydrophobic block chain and the hydrophilic block chain may be adjusted by adjusting conditions in a polymerization reaction, such as a loading ratio between an initiator and a monomer, a reaction time, and a temperature. The chain lengths of the hydrophilic block chain and the hydrophobic block chain (the molecular weight of the amphiphilic block polymer) may be identified by, for example, ¹H-NMR. From the viewpoint of improving the biodegradability of the amphiphilic block polymer, the weight-average molecular weight thereof is preferably 10,000 or less, more preferably 9,000 or less. A chemical crosslink may be formed between the molecules of the amphiphilic block polymer to be used in at least one embodiment of the present invention for the purpose of, for example, accelerating the formation of the gel or improving the stability of the gel.

When the amphiphilic block polymer is mixed with an organic solvent, the amphiphilic block polymer forms an organogel. The amphiphilic block polymer that has formed the organogel self-assembles to form a rod structure in which the hydrophobic block chain is placed inward. When the organic solvent is removed from the amphiphilic block polymer that has formed the organogel, the amphiphilic block polymer forms a xerogel. Even when the xerogel is formed by removing the organic solvent from the amphiphilic block polymer that has formed the organogel, the rod structure may be maintained. Further, when the amphiphilic block polymer that has formed the xerogel and water are mixed with each other, the gel is wetted, and hence the amphiphilic block polymer forms, for example, a hydrosol or a hydrogel. When the amphiphilic block polymer that has formed the xerogel is wetted by the addition of the water, the gel may swell to form a fibrous three-dimensional network. Cells physically aggregate in the three-dimensional network. The amphiphilic block polymer that has formed the hydrosol or the hydrogel is not required to have cell adhesiveness, and preferably has cell non-adhesiveness to aid the following: the cells are repelled from the three-dimensional network formed by the polymer, and hence the cells aggregate. The amphiphilic block polymer according to at least one embodiment of the present invention may be an organogel, may be a xerogel substantially free of any dispersion medium, or may be a hydrosol or a hydrogel wetted with water serving as a dispersion medium. When the cells are cultured in a culture system containing the amphiphilic block polymer, the amphiphilic block polymer is preferably a xerogel substantially free of any dispersion medium, or a hydrosol or a hydrogel wetted with water serving as a dispersion medium from the viewpoint of cytotoxicity. The amphiphilic block polymer according to at least one embodiment of the present invention may be held as a xerogel free of any solvent (dispersion medium), and may be stored at normal temperature.

When the amphiphilic block polymer and the organic solvent are mixed with each other, the amphiphilic block polymer forms the organogel. The organic solvent for forming the organogel is preferably a solvent that easily dissolves the hydrophilic block chain of the amphiphilic block polymer and hardly dissolves the hydrophobic block chain thereof. An organic solvent that dissolves polysarcosine and does not dissolve polylactic acid is preferably used for the amphiphilic block polymer. When such organic solvent is used, the hydrophobic block portions of the molecules of the amphiphilic block polymer aggregate under a mixed state of the amphiphilic block polymer and the organic solvent, and hence a physically crosslinked matrix is easily formed. In addition, when the organogel is formed by using such organic solvent, a xerogel after the removal of the organic solvent also easily has a structure in which the hydrophobic block portions aggregate. Accordingly, it is conceivable that when water is brought into contact with the xerogel, the water easily permeates its hydrophilic block chain portion, and hence a hydrosol or a hydrogel having the same polymer matrix structure as that of the organogel is formed.

The organic solvent to be used in the formation of the organogel is preferably an alcohol having 1 to 6 carbon atoms. Of those, an alcohol having 1 to 4 carbon atoms is preferred because the solubility of the hydrophilic block chain is high, and hence the formation of the xerogel by the removal of the organic solvent is easy. Preferred specific examples of the organic solvent include methanol, ethanol, propanol, 2-propanol, butanol, and 2-butanol.

Two or more kinds of organic solvents may be used as a mixture. The solubility of each of the hydrophobic block chain and the hydrophilic block chain may be adjusted by mixing the two or more kinds of organic solvents. When an organic solvent having low solubility for the hydrophobic block chain is added after the dissolution of the amphiphilic block polymer through use of an organic solvent having high solubility, physical crosslinking by the aggregation of the hydrophobic blocks is accelerated, and hence a gel matrix can be formed. When the two or more kinds of organic solvents are used, at least one kind thereof is preferably the above-mentioned alcohol. Two or more kinds of alcohols may be used. When the organic solvent is a mixed solvent of two or more kinds of organic solvents, the above-mentioned alcohol preferably accounts for 50 wt % or more of the total amount of the organic solvents. The amount of the alcohol with respect to the total amount of the organic solvents is more preferably 60 wt % or more, still more preferably 70 wt % or more.

A ratio between the amphiphilic block polymer and the organic solvent is not particularly limited, and only needs to be set within such a range that the amphiphilic block polymer can be dissolved or swollen in accordance with, for example, the molecular weight of the amphiphilic block polymer and the kind of the organic solvent. From the viewpoint of appropriately keeping a distance between the molecules of the amphiphilic block polymer adjacent to each other to suppress gel formation, the amount of the organic solvent is preferably 100 parts by weight or more and 1,500 parts by weight or less, more preferably 200 parts by weight or more and 1,000 parts by weight or less with respect to 100 parts by weight of the amphiphilic block polymer. The content of the amphiphilic block polymer in the mixture of the amphiphilic block polymer and the organic solvent is preferably 10 wt % or more.

In the formation of the organogel by the amphiphilic block polymer, a viscous liquid having fluidity is preferably prepared as follows: the amphiphilic block polymer and the organic solvent are caused to coexist under heating so that the amphiphilic block polymer may be dissolved or swollen in the organic solvent. The heating activates the molecular motion of the polymer, and hence the swelling and dissolution of the amphiphilic block polymer by the organic solvent are accelerated. A heating temperature only needs to fall within a range equal to or less than the boiling point of the solvent, and is, for example, from about 50° C. to about 95° C., preferably from about 60° C. to about 90° C. When the solution or swollen product of the amphiphilic block polymer is cooled to be equal to or less than its gel point, the formation of a physical crosslink by the hydrophobic block chain is accelerated, and hence an organogel having low fluidity (or free of fluidity) is formed.

The removal of the organic solvent serving as a dispersion medium from the organogel causes the amphiphilic block polymer to form the xerogel (dry gel). A method of removing the organic solvent from the organogel is not particularly limited, and examples thereof include a method including bringing the organogel into contact with a nonsolvent to precipitate the gel, drying with a gas such as nitrogen, vacuum drying, heat drying, heat-vacuum drying, freeze drying, and supercritical drying. For the purpose of, for example, accelerating the removal of the organic solvent, the solvent may be removed after the organogel has been pulverized into particles. The gel may be pulverized while the solvent is removed.

Although the extent to which the organic solvent is removed is not particularly limited, the solvent is preferably removed until the organogel is brought into a solid state free of wettability. It is preferred that the xerogel be substantially free of any dispersion medium. The content of the dispersion medium in the xerogel is preferably 20 wt % or less, more preferably 10 wt % or less, still more preferably 5 wt % or less, and may be 1 wt % or less with respect to the total amount of the xerogel formed by the amphiphilic block polymer. When the organic solvent is sufficiently removed at the time of the formation of the xerogel from the organogel, the content of the organic solvent in the hydrosol or the hydrogel formed from the xerogel is reduced, and hence toxicity to a cell can be reduced, and biosafety can be improved.

When the amphiphilic block polymer that has formed the xerogel and the water are mixed with each other, the amphiphilic block polymer forms the hydrosol or the hydrogel. The hydrogel may include a gel-like portion. In the xerogel obtained by removing the solvent from the organogel, a physically crosslinked structure at the time of the formation of the organogel is easily maintained, and in the hydrosol or the hydrogel obtained by bringing the water into contact with the xerogel to wet the xerogel, the physically crosslinked structure is also easily maintained. When the water is added to wet the xerogel, the amount of the remaining organic solvent can be reduced.

The water to be used at the time of the wetting of the xerogel may be distilled water or an aqueous solution. The aqueous solution is preferably a liquid that does not do damage to cells, such as physiological saline, a buffer, or a medium. A hydrosol or a hydrogel may be formed by adding an aqueous solution having suspended therein the cells to the xerogel. A hydrosol or a hydrogel formed by adding the cells and the medium may be used as it is in cell culture. In this case, there is no need to transplant the cells into the hydrosol or the hydrogel, and the cells can be dispersed in the amphiphilic block polymer. Accordingly, workability is excellent.

The culture system containing the amphiphilic block polymer may contain various substances to be used in cell culture. Examples of the substances to be used in cell culture include various inorganic salts, carbohydrates, amino acids, vitamins, fatty acids, lipids, proteins, and peptides. The culture system containing the amphiphilic block polymer may contain, for example, a cell adhesion epitope, a receptor agonist, a receptor antagonist, a ligand, or an extracellular matrix component. The amphiphilic block polymer may be modified by a functional group having any one of those functions. The culture system may further contain, for example, a preservative, a plasticizer, a surfactant, an antifoamer, a stabilizer, a buffer, a pH adjuster, an osmotic adjuster, or an isotonic agent.

In the preparation of the hydrosol or the hydrogel, a ratio between the amphiphilic block polymer and the water is not particularly limited, and only needs to be set within such a range that the gel can be wet in accordance with, for example, the molecular weight and mass of the amphiphilic block polymer. From the viewpoint of appropriately keeping a distance between the molecules of the amphiphilic block polymer adjacent to each other, the amount of the water to be mixed is preferably 50 parts by weight or more and 1,500 parts by weight or less, more preferably 100 parts by weight or more and 1,000 parts by weight or less with respect to 100 parts by weight of the amphiphilic block polymer. In the hydrosol, the content of the amphiphilic block polymer in the mixture of the water and the amphiphilic block polymer is preferably 0.1 wt % or more. In the hydrogel, the content of the amphiphilic block polymer in the mixture of the water and the amphiphilic block polymer is preferably 10 wt % or more.

After the formation of the hydrosol or the hydrogel, a xerogel may be formed by removing the water. In, for example, the case where a substance insoluble in an organic solvent or a substance that is liable to be degraded by the organic solvent is incorporated into the amphiphilic block polymer, when such substance is mixed into the amphiphilic block polymer that has formed the hydrosol or the hydrogel, and then the water is removed, a xerogel containing such substance is formed. When the formed xerogel is wetted with water again, a hydrosol or a hydrogel is formed.

From the viewpoint of reducing toxicity and stimulation to a living body, the content of the organic solvent in the hydrosol or the hydrogel is preferably as small as possible. The ratio of the water to the entirety of the dispersion medium of the amphiphilic block polymer that has formed the hydrosol or the hydrogel is preferably 80 wt % or more, more preferably 90 wt % or more, still more preferably 95 wt % or more, particularly preferably 98 wt % or more. To reduce the content of the organic solvent, the ratio at which the organic solvent is removed at the time of the formation of the xerogel from the organogel is preferably increased. The content of the organic solvent can also be reduced by repeatedly performing, after the formation of the xerogel from the organogel, the formation of a hydrosol or a hydrogel, and the formation of a xerogel by the removal of the dispersion medium.

The organoid composition according to at least one embodiment of the present invention may be recovered under the state of being included in the three-dimensional network of the amphiphilic block polymer, or the cell cluster in the organoid composition may be recovered by degrading the three-dimensional network of the amphiphilic block polymer. When water is added to the amphiphilic block polymer that has formed gel to reduce the concentration of the amphiphilic block polymer, an interaction between the molecules of the polymer adjacent to each other is weakened, and hence the three-dimensional network of the amphiphilic block polymer is easily degraded. The cell cluster may be recovered by, for example, culturing its cells in a hydrosol, and then adding moisture such as a medium to the hydrosol. The cell cluster may also be recovered by subjecting the three-dimensional network of the amphiphilic block polymer to degradation and centrifugation through pipetting or the like.

The organoid composition according to at least one embodiment of the present invention may be appropriately subcultured. The composition may be passaged, for example, as follows: the composition is suspended in a liquid containing at least any one of, for example, a protease, such as trypsin, a collagenase, or Dispase I, EDTA, and EGTA, for example, TrypLE Select™ (Thermo Fisher Scientific); the suspension is incubated at 37° C.; pipetting, centrifugation, and the like are each performed a plurality of times, followed by the removal of a supernatant to provide a cell cluster; the cell cluster is suspended in a medium or the like; and the suspension is added to a culture system containing a new amphiphilic block polymer. For example, a medium to be used in the passage of an organoid may be used as the medium to be used in the passage of the organoid composition.

### (Utilization of Organoid Composition)

As described above, in the endodermal cells in the organoid composition according to at least one embodiment of the present invention, the expression level of the pharmacokinetics-related gene increases as compared to that in the two-dimensionally cultured endodermal cell-derived amphiphilic block polymer-cultured organoid. In addition, the organoid composition according to at least one embodiment of the present invention shows high activity not only for the pharmacokinetics-related gene but also for a drug-metabolizing enzyme. Thus, the composition can be effectively utilized in an in vitro pharmacokinetic evaluation. Further, the organoid composition according to at least one embodiment of the present invention shows excellent sensitivity to a drug causing hepatotoxicity. Thus, the organoid composition according to at least one embodiment of the present invention can be effectively utilized in a drug toxicity evaluation. The present invention also covers an organoid composition for evaluating pharmacokinetics and/or evaluating drug toxicity, the composition including the organoid composition. According to the method of producing an organoid composition according to at least one embodiment of the present invention, an organoid composition having such excellent performance can be supplied. The excellent organoid composition or a cell population thereof may be used as a kit for evaluating pharmacokinetics and/or evaluating drug toxicity. Thus, the hepatopathy risk of a compound can be predicted at an early stage in the research and development of a medicine such as drug discovery and development, and the prediction is extremely useful in increasing the success rate of the research and development, and curtailing cost therefor and the period thereof. The present invention also covers a kit for evaluating pharmacokinetics and/or evaluating drug toxicity, the kit including: the organoid composition; and a device and/or a reagent required for a pharmacokinetic evaluation and/or a drug toxicity evaluation or inspection. Further, the present invention covers a method of evaluating pharmacokinetics and/or a method of evaluating drug toxicity, the method including using the organoid composition.

The organoid composition according to at least one embodiment of the present invention may be used in regenerative medicine or cell therapy in, for example, a case in which cell transplantation has heretofore been required. As described above, the organoid composition according to at least one embodiment of the present invention is characterized by including the amphiphilic block polymer having the hydrophilic block chain having the sarcosine unit and the hydrophobic block chain having the lactic acid unit. Thus, the composition is excellent in safety, and hence an effective therapeutic effect can be expected.

### EXAMPLES

The present invention is specifically described below by way of Reference Examples and Examples for a better understanding of the present invention. Needless to say, however, Reference Examples and Examples are not intended to limit the scope of the present invention.

### (Reference Example 1) Establishment of Human Liver Organoid

In this Reference Example, a human liver organoid was established by using frozen primary human liver cells. Commercial frozen primary human liver cells (HC4-24; XenoTech) were used in the establishment of the human liver organoid. The liver cells were suspended in 50 μL of Matrigel (Corning) so that their number became 2×10⁴ cells, followed by seeding on a 24-well plate at 50 μL/well. The plate was left at rest at 37° C. for 15 minutes so that the Matrigel was caused to gel. A medium for a human liver organoid described in a previous report (Broutier et al., Nat Protoc., 2016-9-11(9): 1724-43) was added to the resultant, and then organoid culture was performed by replacing the medium with a new one once per 3 days. The composition of the medium for a human liver organoid is as follows: to a DMEM/F-12 medium (Thermo Fisher Scientific), 10% R-spondinl conditional medium (homemade), 1×B27 supplement Minus Vitamin A (Thermo Fisher Scientific), 1% penicillin/streptomycin (Nacalai tesque), 10 mM nicotinamide (Sigma), 1 mM N-acetyl-L-cysteine (Sigma), 1×GlutaMAX (Thermo Fisher Scientific), 10 mM HEPES (Nacalai tesque), 50 ng/mL EGF (R&D Systems), 10 nM gastrin (Merck), 25 ng/mL HGF (R&D Systems), 100 ng/mL FGF10 (Peprotech), 5 mM A83-01 (FUJIFILM Wako Pure Chemical), and 10 mM forskolin (FUJIFILM Wako Pure Chemical) were added. The organoid was passaged every 7 to 10 days at a ratio of from 1:3 to 1:5. In each of Examples, human liver organoids in a first passage to a 15th passage after the establishment were used. The human liver organoid produced in Reference Example 1 is referred to as “Matrigel-cultured human liver organoid” in Examples below.

### (Reference Example 2) Culture of Primary Human Liver Cell

In this Reference Example, 1 lot (lot name: HC4-24, XenoTech) of cultured primary human liver cells were cultured. The cells were rapidly thawed in a shaking water bath at 37° C., and were suspended in OptiTHAW Hepatocyte Isolation Kit (XenoTech) that had been preheated, followed by centrifugation at 100 g and room temperature for 5 minutes. The liver cells were suspended in HCM (Lonza) so that their density became 2.5×10⁵ cells/cm², followed by seeding on 48-well Corning^{R}BioCoat™ Collagen I-coated Microplates (Corning). After the cells had been cultured for 48 hours, various tests were performed.

### (Reference Example 3) Establishment of Human Intestinal Organoid

In this Reference Example, an intestinal organoid was established by using a human duodenal biopsy specimen.

### (Human Duodenal Biopsy)

Human duodenal biopsy was performed on a patient (one person) for whom approval had been obtained from an ethics committee in Hokkaido Public University Corporation Sapporo Medical University and from whom consent had been obtained during upper gastrointestinal endoscopy. In a duodenal region in which no inflammatory findings were present under endoscopic observation, two to four biopsy specimens were collected from a lamina propria mucosae with a bioptome. The resultant biopsy specimens were stored in ice-cold phosphate-buffered saline (PBS) containing 1×antibiotic-antimycotic (Thermo Fisher Scientific) until an organoid-establishing operation was performed.

### (Establishment of Human Intestinal Organoid)

A human duodenal organoid was established by using a method obtained by modifying some points in previous reports (Sato, T. et al., Gastroenterology. 2011 November; 141(5): 1762-72, and Sugimoto, S., and Sato, T., Methods Mol Biol. 2017; 1612: 97-105). First, the resultant biopsy specimens were each incubated at 4° C. for 30 minutes while being gently stirred in 2.5 mM ethylenediaminetetraacetic acid (EDTA, Thermo Fisher Scientific), followed by vigorous pipetting to isolate a crypt from a tissue. The crypt was recovered in a tube. Next, the recovered crypt was resuspended in Matrigel (Corning), and 25 μL to 40 μL of the suspension was added to the center of each well of a 24-well plate (Thermo Fisher Scientific). Finally, the plate was incubated at 37° C. for from 10 minutes to 15 minutes so that the Matrigel was caused to gel. 400 Microliters of IntestiCult Organoid Growth Medium (Human) (STEMCELL Technologies) containing 1×antibiotic-antimycotic and 10 μM Y-27632 (FUJIFILM Wako Pure Chemical) was added to each well to establish the human duodenal organoid.

### (Maintenance of Human Intestinal Organoid)

A human intestinal organoid was maintained by using Advanced DMEM/F12 containing penicillin/streptomycin, 10 mmol/L HEPES, GlutaMAX, 1×N2 Supplement (Thermo Fisher Scientific), 1×B27 Supplement (Thermo Fisher Scientific), 1 mM N-acetylcysteine (Merck), 10 mM nicotinamide (Merck), 50% Afamin-Wnt3A CM, 10% R-Spondinl CM, 500 nM A83-01 (FUJIFILM Wako Pure Chemical), 10 μM SB202190 (Merck), 50 ng/mL human EGF (Thermo Fisher Scientific), 100 ng/mL human Noggin (Peprotech), and 10 nM [Leu15]-Gastrin1 (Merck). To maintain the human intestinal organoid, the medium was replaced with a new one every 2 days, and the organoid was passaged once per 1 to 2 weeks. A procedure for the passage of the human intestinal organoid is based on previous reports (Sato, T. et al., Gastroenterology. 2011 November; 141(5): 1762-72, Sugimoto, S., and Sato, T., Methods Mol Biol. 2017; 1612: 97-105, and Miyoshi, H., and Stappenbeck, T.S., Nat Protoc. 2013 December; 8 (12): 2471-82), but slight modification has been added thereto. Each well was washed with PBS containing 1×antibiotic-antimycotic, and an organoid-cultured product was scraped off and suspended in the PBS containing 1×antibiotic-antimycotic. After the suspension had been vertically pipetted 5 to 10 times, the suspension was passed through a 70-micrometer strainer and resuspended in Matrigel so that a desired concentration was obtained. Next, 40 μL to 50 μL of the organoid suspension was applied to the center of each well of a 24-well plate. The Matrigel was polymerized at 37° C. for 10 minutes, and 400 μL/well of an organoid culture solution was added thereto. The human intestinal organoid produced in Reference Example 3 is referred to as “Matrigel-cultured human intestinal organoid.”

### (Example 1) Production of Human Liver Organoid Composition including HYDROX and Liver Function Evaluation Thereof

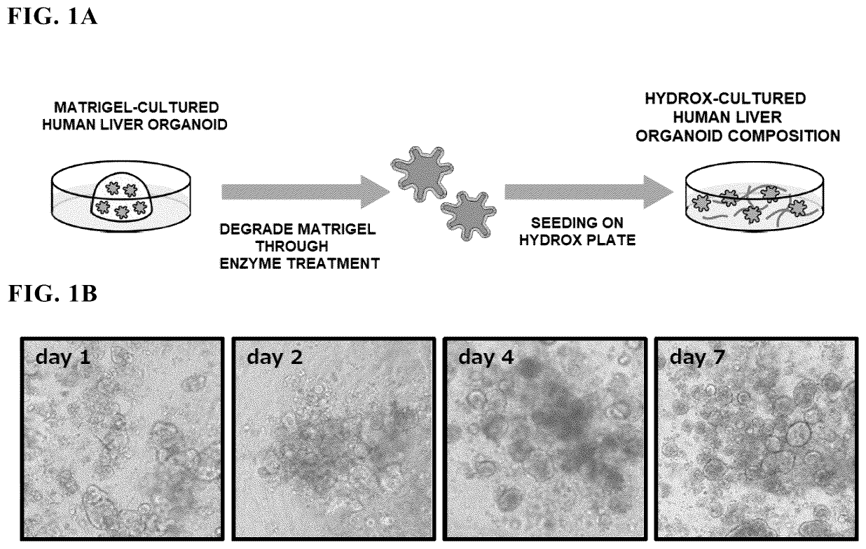

In this Example, the human liver organoid produced in Reference Example 1 was cultured with HYDROX (FIG. 1A) to produce a human liver organoid composition including the HYDROX, and its liver function was identified. The Matrigel-cultured human liver organoid produced in Reference Example 1 was treated with TrypLE Select so that the Matrigel was degraded. The resultant cell cluster was suspended in the medium for a human liver organoid, and was seeded on a HYDROX plate at 300 μL/well, followed by three-dimensional culture (HYDROX culture). The cell cluster was reseeded on a new HYDROX plate every 7 days. The HYDROX plate was produced as described below. In a 48-well adhesive plate, a HYDROX raw material polymer (Psar-PLLA) was mixed with 95% ethanol (Nacalai tesque) at a concentration of 10 mg/mL, and the mixture was warmed at 70° C. for 10 minutes. The dissolved polymer solution was dropped into a culture plate, and was dried for 8 hours or more to produce the human liver organoid composition including the HYDROX. In this Example and the following Examples, the human liver organoid composition including the HYDROX is referred to as “HYDROX-cultured human liver organoid composition.”

The HYDROX-cultured human liver organoid composition was observed with a phase-contrast microscope on day 1, 2, 4, or 7 (FIG. 1B). Cell aggregation was observed from the first day of the culture, and thereafter, organoid formation was observed. In other words, the human liver organoid was able to be three-dimensionally cultured with the HYDROX.

Subsequently, to evaluate the properties of the HYDROX-cultured human liver organoid composition, the analysis of the expression level of a liver cell marker gene in the HYDROX-cultured human liver organoid composition on a 7th, 14th, or 21st day after the start of the HYDROX culture was performed by a qRT-PCR method. A gene expression level in the Matrigel-cultured human liver organoid was defined as 1.0. All data are represented as mean±S.D. (n=3). Specifically, the analysis was performed by the following procedure. Total RNA was extracted from each cell population with ISOGEN (NIPPON GENE). cDNA was synthesized from 500 ng of each total RNA by performing a reverse transcription reaction through use of a Superscript VILO cDNA synthesis kit (Thermo Fisher Scientific). A quantitative RT-PCR was performed by using SYBR Green PCR Master Mix (Applied Biosystems) in a StepOnePlus real-time PCR system (Applied Biosystems). A target mRNA expression level was relatively determined by using a 2-ΔΔOCT method. Glyceraldehyde 3-phosphate dehydrogenase (GAPDH) was used as an internal standard gene. The sequence of a primer used in the quantitative RT-PCR was obtained from PrimerBank (https://pga.mgh.harvard.edu/primerbank/).

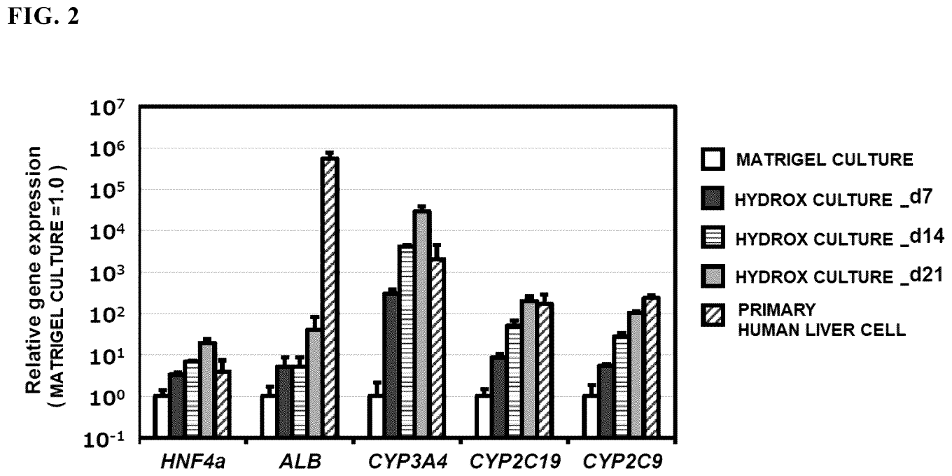

As a result, in a HYDROX-cultured group, the expression levels of many liver cell marker genes significantly increased as compared to those in a Matrigel-cultured group. In addition, the expression levels were comparable to those of primary human liver cells (FIG. 2).

Next, the substrates of various drug-metabolizing enzymes were each caused to act on the human liver organoid composition on the 7th, 14th, or 21st day after the start of the HYDROX culture, or the primary human liver cell, and the activity of each of the drug-metabolizing enzymes, which was an important function of a liver cell, was measured by determining the amount of a metabolite in a culture supernatant 24 hours after the action with an UPLC MS/MS. All data are represented as mean±S.D. (n=3). Specifically, UPLC MS/MS analysis was performed for measuring CYP3A4, CYP2C19, CYP1A2, or CYP2C8 activity. The substrate (1 μM Midazolam, 80 μM (S)-(+)-Mephenytoin, 0.5 μM Ethoxyresorufin, or 50 nM Amodiaquine) of each of the drug-metabolizing enzymes was caused to act on the cell, and a supernatant was recovered 24 hours thereafter. A sample obtained by mixing the supernatant with acetonitrile (FUJIFILM Wako Pure Chemical) whose amount was equal to that of the supernatant was filtered with AcroPrep Advance 96-Well Filter Plates (Pall Corporation), and the content of its metabolite was measured with a mass spectrometer (Xevo TQ-S, Waters Corp., Milford, USA) connected to an UPLC (ACQUITY UPLC, Waters). A BEH C18 column (1.7 μm, 2.1×50 mm, Waters) was used in LC separation. The LC separation was performed in a gradient profile in which a mobile phase including a solvent A (0.1% formic acid/water) and a solvent B (0.1% formic acid/acetonitrile) was flowed at a flow rate of 1.0 ml/min. Gradient conditions are as follows: 0 min-2% B, 1.0 min-95% B, 1.25 min-95% B, 1.26 min-2% B, and 1.75 min-2% B. 5 Microliters of the sample was injected into the column, and a compound concentration was calculated on the basis of a standard curve.

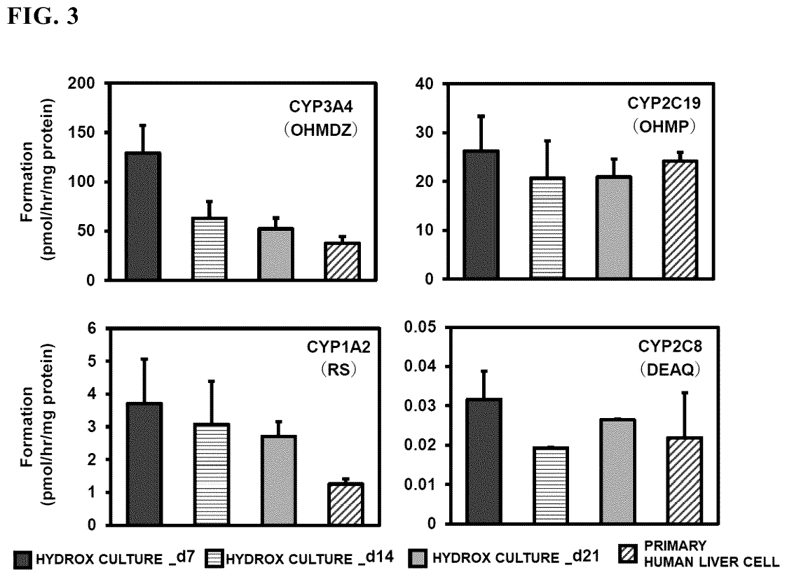

An enzyme-specific substrate was caused to act on the HYDROX-cultured human liver organoid composition, and the amount of its metabolite was determined. As a result, it was shown that the HYDROX-cultured human liver organoid composition had high drug-metabolizing enzyme activity at the same level as that of the primary human liver cell (FIG. 3).

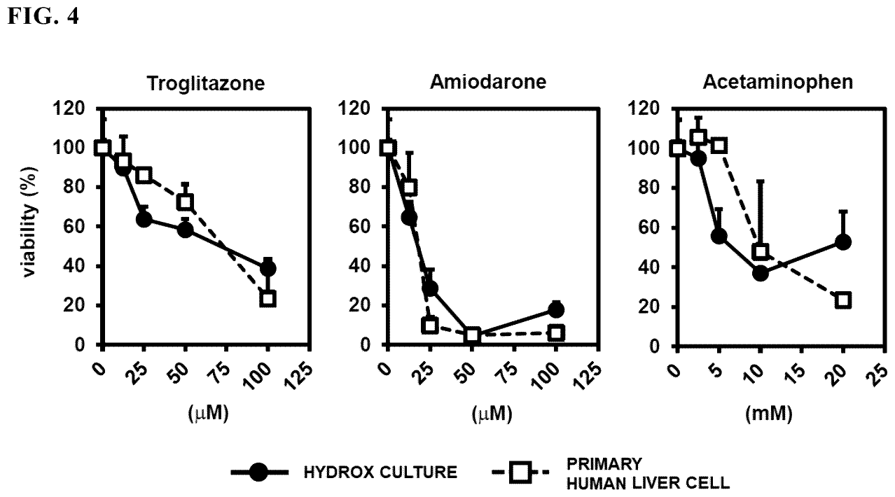

Further, at the initial stage of the development of a new drug, the safety of the drug needs to be accurately evaluated by a hepatotoxicity evaluation test. Accordingly, an investigation was made on the potential of the HYDROX-cultured human liver organoid composition to be applied to the hepatotoxicity evaluation test. Troglitazone, amiodarone, and acetaminophen known as drugs each causing hepatotoxicity were each caused to act on the HYDROX-cultured human liver organoid composition or the primary human liver cell, and a cell viability on a 7th day was measured. As a result, the viability reduced in accordance with the concentration of each of the drugs, and the extents of the reduction were similar between the cells (FIG. 4). It was shown from the foregoing investigation that the HYDROX was applicable to a human liver organoid, and hence enabled the production of a high-function liver organoid composition, which had a function close to that of a living body and was applicable to drug discovery research.

### (Example 2) Production of Human Intestinal Organoid Composition including HYDROX and Evaluations of Properties Thereof

In this Example, a human intestinal organoid composition was produced by using the HYDROX, and the organoid composition was evaluated.

Cells recovered by the same procedure as that for the passage of a human intestinal organoid described in Reference Example 3 were suspended in a medium for a human intestinal organoid, and were seeded on a HYDROX plate at 500 μL/well, 250 μL/well, or 100 μL/well, followed by three-dimensional culture (HYDROX culture). Thus, a human intestinal organoid composition including the HYDROX was produced. The HYDROX plate was produced as described below. A HYDROX raw material polymer (Psar-PLLA) was mixed with 95% ethanol (Nacalai tesque) at a concentration of 10 mg/mL, and the mixture was warmed at 70° C. for 10 minutes. The dissolved polymer solution was dropped into a 24-well, 48-well, or 96-well culture plate, and was dried for 8 hours or more. In this Example and the following Examples, the human intestinal organoid composition including the HYDROX is referred to as “HYDROX-cultured human intestinal organoid composition.”

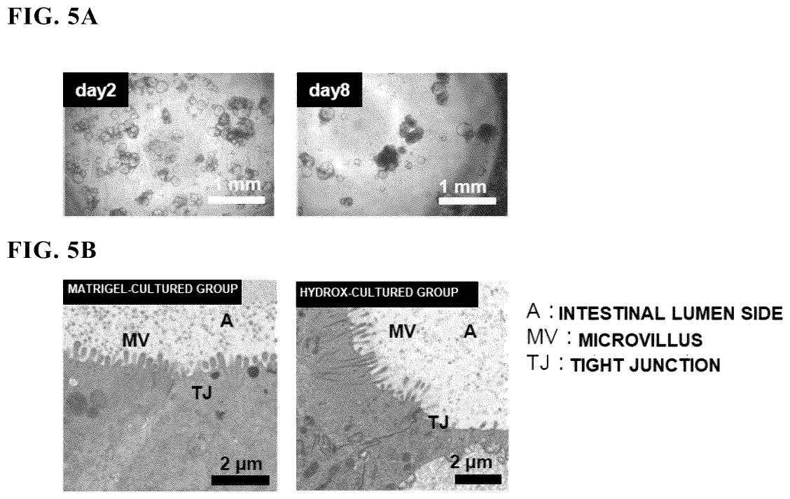

To start the HYDROX culture, the cell suspension of the Matrigel-cultured human intestinal organoid was seeded on a HYDROX plate, and the organoid was observed with a phase-contrast microscope on the 2nd day and 8th day of the culture. As time passed, cell aggregation caused by the HYDROX was observed, and hence organoid formation was observed (FIG. 5A). In addition, to perform a morphological evaluation, a Matrigel-cultured group and a HYDROX-cultured group were observed with a transmission electron microscope. In each of the Matrigel-cultured group and the HYDROX-cultured group, structures characteristic of an intestinal epithelium, such as a microvillus and a tight junction, were observed (FIG. 5B). In other words, the human intestinal organoid was able to be three-dimensionally cultured with the HYDROX.

Subsequently, to evaluate the properties of the HYDROX-cultured human intestinal organoid composition, the analysis of the expression level of an intestinal differentiated cell marker gene and the immunofluorescence staining thereof were performed. The expression level of the intestinal differentiated cell marker gene was determined by the same approach as that of Example 1, that is, a quantitative RT-PCR. A gene expression level in a human small intestine was defined as 1.0. All data are represented as mean±S.D. (n=3).

### (Immunofluorescence Staining)

Proteins CHGA and E-cad in each of the HYDROX-cultured human intestinal organoid composition and the Matrigel-cultured human intestinal organoid were subjected to immunofluorescence staining. DAPI represents a nucleus. Cells recovered by the same procedure as that for the passage of a human intestinal organoid were embedded in Tissue-Tek O.C.T. Compound (Sakura Finetek Japan) and rapidly frozen to form a frozen block on the Tissue-Tek Cryomold (Sakura Finetek Japan). A frozen section was cut out on a microscope slide with a rotary cryostat microtome CM1950 (Leica Biosystems). 4% Paraformaldehyde Phosphate Buffer Solution (FUJIFILM Wako Pure Chemical) was added to the cells on the microscope slide, and the mixture was incubated at room temperature for 15 minutes so that the cells were immobilized. Next, PBS (blocking buffer) containing 2% bovine serum albumin (BSA) (Nacalai tesque) and 0.2 vol % Triton X-100 (Merck) was added to the resultant, and the mixture was subjected to blocking at room temperature for 15 minutes. After that, a blocking buffer containing a primary antibody (Table 1) was added to the blocked product, and the mixture was incubated at 4° C. overnight. Finally, a blocking buffer containing a secondary antibody (Table 1) was added to the incubated product, and the mixture was incubated at room temperature for 1 hour. The incubated product was subjected to observation. Nuclear staining was performed with 4′,6-diamidino-2-phenylindole (DAPI) (Nacalai tesque). An image was taken with a confocal laser scanning microscope FV10i (Olympus).

TABLE 1

| Name | Source | Cat no |
|---|---|---|
| Anti-E Cadherin | Abcam | ab40772 |
| antibody | | |
| Anti-Chromogranin A | Abcam | ab715 |
| antibody | | |
| Donkey anti-Mouse | Thermo Fisher | A-21202 |
| IgG (H + L) Highly | Scientific | |
| Cross-Adsorbed | | |
| Secondary Antibody, | | |
| Alexa Fluor 488 | | |
| Donkey anti-Rabbit | Thermo Fisher | A-21207 |
| IgG (H + L) Highly | Scientific | |
| Cross-Adsorbed | | |
| Secondary Antibody, | | |
| Alexa Fluor 594 | | |

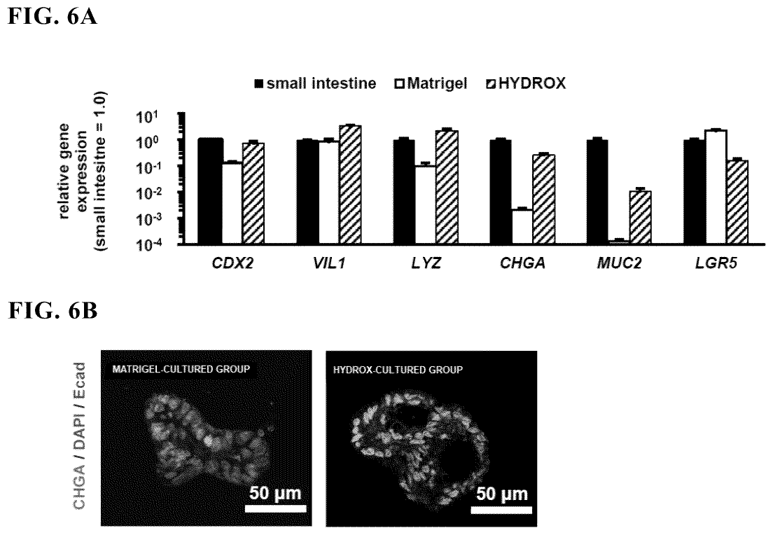

As a result, in the HYDROX-cultured group, the expression levels of many intestinal differentiated cell marker genes increased as compared to those in the Matrigel-cultured group, and the expression levels were comparable to those of a human small intestine. In addition, the expression of an intestinal cell marker gene LGR5 reduced (FIG. 6A). Further, it was revealed that the HYDROX-cultured human intestinal organoid composition was a cultured body formed of a columnar epithelium as in the Matrigel group. In the HYDROX-cultured human intestinal organoid composition, the expression of an enteroendocrine cell marker CHGA whose expression could not have been recognized in the Matrigel group was able to be recognized (FIG. 6B).

Next, to evaluate a molecule related to the pharmacokinetics of the HYDROX-cultured human intestinal organoid composition, the analysis of the expression levels of pharmacokinetics-related genes and the measurement of the activity of the most important drug-metabolizing enzyme CYP3A4 in the HYDROX-cultured human intestinal organoid composition were performed. The expression levels of the pharmacokinetics-related genes in the human intestinal organoid composition were analyzed by the same approach as that of Example 1, that is, a quantitative RT-PCR. A gene expression level in a human small intestine was defined as 1.0. All data are represented as mean±S.D. (n=3). The activity of CYP3A4 was measured in the same manner as in Example 1 except that in the approach of Example 1, Midazolam was caused to act on the cell, and a supernatant was recovered 60 minutes thereafter instead of the recovery of the supernatant 24 hours thereafter. All data are represented as mean±S.D. (n=3).

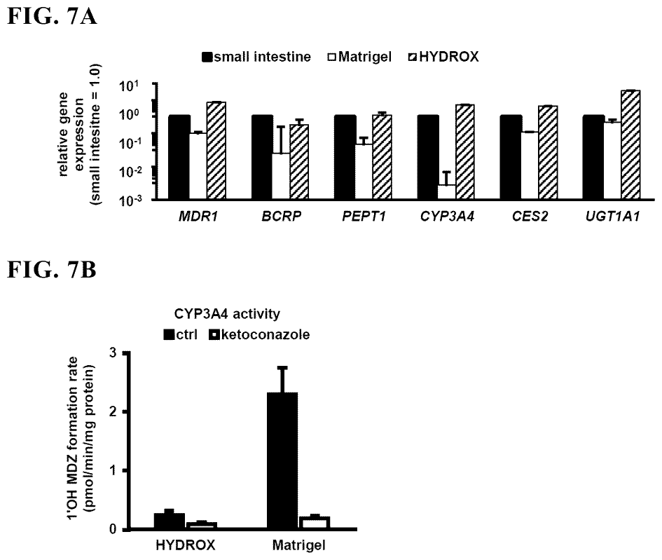

In the HYDROX-cultured group, the expression of a main drug transporter or drug-metabolizing enzyme was improved as compared to that in the Matrigel-cultured group, and hence an expression level comparable to or more than that in the human small intestine was shown (FIG. 7A). In addition, it was found from the result of the measurement of CYP3A4 metabolism activity that the HYDROX-cultured group showed activity about 10 times as high as that of the Matrigel group (FIG. 7B). It was shown from the foregoing investigation that the HYDROX was applicable to a human intestinal organoid, and hence enabled the production of a high-function human intestinal organoid, which had properties closer to those of a living body and was able to be expected to be applied to drug discovery research.

### (Example 3) Comparison Between Liver Organoid Composition and Two-dimensionally Cultured Endodermal Cell-Derived Amphiphilic Block Polymer-Cultured Liver Organoid

In this Example, the expression levels of pharmacokinetics-related genes in the liver organoid composition produced in Example 1 and a two-dimensionally cultured endodermal cell-derived amphiphilic block polymer-cultured liver organoid were compared to each other.

### (Production of Two-dimensionally Cultured Endodermal Cell-Derived Amphiphilic Block Polymer-Cultured Liver Organoid)

The two-dimensionally cultured endodermal cell-derived amphiphilic block polymer-cultured liver organoid (2D-derived HYDROX-applied liver organoid) was produced by a method described in J. Enomoto et al., ACS Applied Bio Materials, 2021, Sep. 20; 4(9): 7290-7299. Specifically, human iPS cells (Tic strain) were cultured in a RPMI 1640 medium (Sigma) containing 100 ng/mL activin A (R&D Systems), 1×GlutaMAX (Thermo Fisher Scientific), and 1×B27 supplement Minus Vitamin A (Thermo Fisher Scientific) for 4 days to be differentiated into definitive endoderm cells. Next, the definitive endoderm cells were cultured in a RPMI 1640 medium containing 20 ng/mL bone morphogenetic protein 4 (BMP4; R&D Systems), 20 ng/mL fibroblast growth factor 4 (FGF4; R&D Systems), 1×GlutaMAX (Thermo Fisher Scientific), and 1×B27 supplement Minus Vitamin A (Thermo Fisher Scientific) for 5 days to be differentiated into hepatoblast-like cells. Next, the hepatoblast-like cells were cultured in a RPMI 1640 medium containing 20 ng/mL hepatocyte growth factor (HGF; R&D Systems), 1×GlutaMAX (Thermo Fisher Scientific), and 1×B27 supplement Minus Vitamin A (Thermo Fisher Scientific) for 5 days.

TrypLE Select™ was caused to act on the hepatoblast-like cells on the 14th day of the culture to detach the cells, and single cells were recovered. In a HYDROX plate, the recovered cells were seeded into a hepatocyte culture medium (HCM; Lonza) having added thereto 20 ng/mL oncostatin M (OsM; R&D Systems) and 3×GlutaMAX (Thermo Fisher Scientific) at a density of 5×10⁵ cells/0.5 mL/24 wells, and were cultured for 11 days. Thus, the two-dimensionally cultured endodermal cell-derived amphiphilic block polymer-cultured liver organoid was produced. The HYDROX plate was produced by the same approach as that of each of Examples 1 and 2.

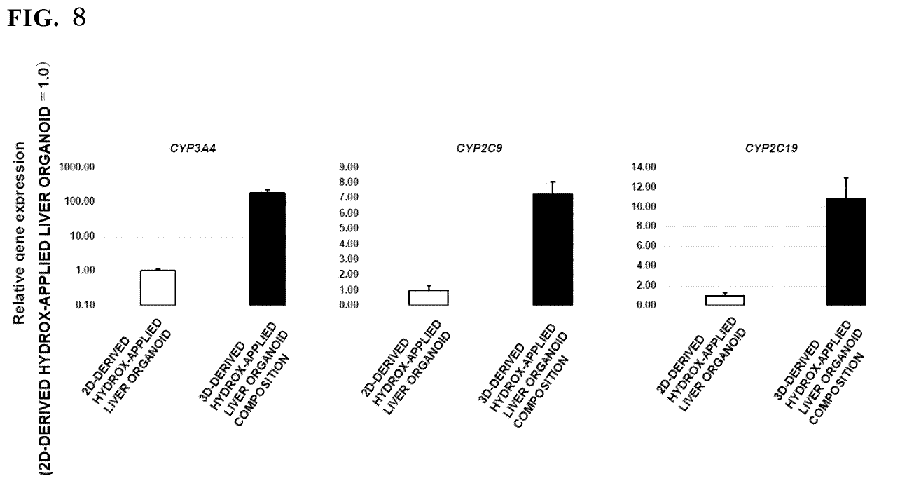

The expression levels of the pharmacokinetics-related genes in the liver organoid composition on the 7th day after the start of the HYDROX culture and the two-dimensionally cultured endodermal cell-derived amphiphilic block polymer-cultured liver organoid on the 11th day after the start of the HYDROX culture were analyzed by the same approach as that of Example 1, that is, a quantitative RT-PCR. It was shown that the expression levels of the pharmacokinetics-related genes in the liver organoid composition increased as compared to the expression levels of the pharmacokinetics-related genes in the two-dimensionally cultured endodermal cell-derived amphiphilic block polymer-cultured organoid (FIG. 8).

### (Example 4) Recognition of Coexistence of HYDROX-Cultured Organoid and Amphiphilic Block Polymer

In this Example, whether or not an amphiphilic block polymer coexisted in each of the organoid compositions produced in Examples 1 and 2 was investigated.

The total amount of each of the organoid compositions on the 7th day of the culture (day 7) produced in Examples 1 and 2 was passaged to a new HYDROX plate. Three days after the passage, each organoid composition was recovered on day 10, and was centrifuged, followed by the removal of a supernatant. After that, each organoid composition was suspended in PBS and centrifuged, followed by the washing of the organoid composition. The step of suspending the composition in PBS, followed by centrifugation was performed a total of three times. After the removal of the supernatant, a precipitated cell cluster was freeze-dried for 4 days, and was then subjected to 1H-NMR measurement through use of deuterated methanol (CD₃OD) as a solvent at a temperature of 25° C. Each cell cluster and conditions for the ¹H-NMR measurement are shown in Table 2. Each organoid composition was subjected to cotton-plug filtration treatment.

TABLE 2

| | | | HYDROX-cultured |
|---|---|---|---|
| | | HYDROX-cultured | human liver |
| | | human intestinal | organoid |
| | | organoid | composition- |
| | | composition- | derived cell |
| | | derived cell | pellet |
| Appearance | | Opaque solid | Opaque solid |
| Weight | | 2.9 mg | 1.8 mg |
| Conditions | Deuterated solvent | CD₃OD | |
| for NMR | Measurement | 25° C. | |
| measurement | temperature | | |
| (400 MHz) | Number of scans | 256 | 512 |

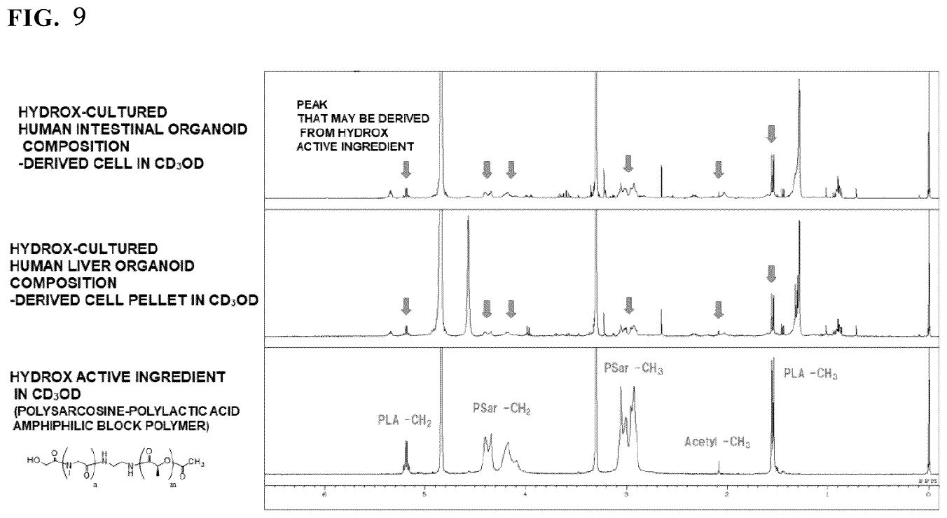

It is conceivable from the foregoing results that the cells cultured by using the HYDROX each contain a HYDROX active ingredient (polysarcosine-polylactic acid amphiphilic block polymer) (FIG. 9). A NMR peak may reduce owing to the loss of the HYDROX active ingredient (polysarcosine-polylactic acid amphiphilic block polymer) during, for example, the cell culture and the washing treatment.

The organoid composition according to at least one embodiment of the present invention is excellent in safety because the composition is characterized by including the amphiphilic block polymer having the hydrophilic block chain having the sarcosine unit and the hydrophobic block chain having the lactic acid unit. Thus, the composition is particularly applicable to regenerative medicine or cell therapy, and hence an effective therapeutic effect can be expected. According to regenerative medicine using the organoid composition according to at least one embodiment of the present invention, an organoid composition having higher immune compatibility for a patient can be selected, and a risk involved in organ transplantation can be reduced. Accordingly, the composition is extremely excellent.

In the organoid composition according to at least one embodiment of the present invention, the expression level of a pharmacokinetics-related gene increases as compared to that in an organoid produced from a two-dimensional endodermal cell. Further, the organoid composition according to at least one embodiment of the present invention shows high activity not only for the expression of the pharmacokinetics-related gene but also for a drug-metabolizing enzyme. Further, the composition shows excellent sensitivity to a drug causing hepatotoxicity. Thus, the organoid composition according to at least one embodiment of the present invention can be effectively utilized in a pharmacokinetic evaluation and/or a drug toxicity evaluation.

What is claimed is:

1. An organoid composition, comprising:
an organoid including a cell cluster of endodermal cells; and
an amphiphilic block polymer present in the cell cluster for forming the organoid, the polymer having a hydrophilic block chain having a sarcosine unit and a hydrophobic block chain having a lactic acid unit.

2. The organoid composition according to claim 1, wherein the amphiphilic block polymer has the hydrophilic block chain having the 20 or more sarcosine units and the hydrophobic block chain having the 10 or more lactic acid units.

3. The organoid composition according to claim 1, wherein the endodermal cells are one of liver cells or intestinal cells.

4. The organoid composition according to claim 3, wherein the liver cells are one of liver cells collected from a living body or pluripotent stem cell-derived liver cells.

5. The organoid composition according to claim 3, wherein the intestinal cells are one of intestinal epithelial cells collected from a living body or pluripotent stem cell-derived intestinal epithelial cells.

6. An organoid composition for evaluating pharmacokinetics and/or evaluating drug toxicity, comprising the organoid composition of claim 1.

7. A method of producing the organoid composition of claim 1, comprising the following steps:
(1) a step of dispersing endodermal cells; and
(2) a step of culturing the dispersed cells in a culture system containing an amphiphilic block polymer having a hydrophilic block chain having a sarcosine unit and a hydrophobic block chain having a lactic acid unit.

8. The production method according to claim 7, wherein the step of dispersing the endodermal cells serving as the step (1) is a step of dispersing the endodermal cells that have three-dimensionally aggregated.

9. The production method according to claim 8, wherein the endodermal cells are one of liver cells or intestinal cells.

10. A kit for evaluating pharmacokinetics and/or evaluating drug toxicity, comprising:
the organoid composition of claim 1; and
a device and/or a reagent required for a pharmacokinetic evaluation and/or a drug toxicity evaluation or inspection.

11. A method of evaluating pharmacokinetics and/or a method of evaluating drug toxicity, comprising using the organoid composition of claim 1.
